(19) United States
(12) Patent Application Publication
(10) Pub. No.: US 2005/0222625 A1
(43) Pub. Date: Oct. 6, 2005

(54) Method and apparatus for non-invasive therapy of cardiovascular ailments using weak pulsed electromagnetic radiation

(76) Inventors: Shlomo Laniado, Tel Aviv (IL); Zvi Kamil, Tel Aviv (IL); Eli Nhaissi, Old Westbury, NY (US)

Correspondence Address: Dennis M. Flaherty, Esq.;Ostrager Chong Flaherty & Broitman P.C., Suite 825, 250 Park Avenue, New York, NY, 10177-0899, US

(21) Appl. No.: 10/910,163

(22) Filed: Aug. 2, 2004

Related U.S. Application Data
(60) Provisional application No. 60/558,336, filed on Mar. 30, 2004.
(60) Provisional application No. 60/587,085, filed on Jul. 12, 2004.

(51) Int. Cl. A61N 1/00

(52) U.S. Cl. 607/2

(57) ABSTRACT

A method and an apparatus for the treatment of cardiac hypertrophic heart failure, hypertropic cardiomyopathy, atrial or ventricular brady-arrhythmias (slow heart rate), atrial flutter-fibrillation and similar cardiac ailments, as well as peripheral vascular disease and hypertension, using a weak pulsed magnetic field or a very weak magnetic field. A transducer that emits weak electromagnetic radiation is placed on the patient's chest or legs and, as a result the very weak electromagnetic field can cause activation, reactivation, inhibition or remodeling of electrophysiological change in cardiac tissue in an irradiated heart or vessels. This treatment method has wide application for use in patients with various heart and vascular ailments.

### RELATED PATENT APPLICATION

This application claims the benefit, under Title 35, United States Code, § 119(e), of U.S. Provisional Application No. 60/558,336 filed on Mar. 20, 2004, and U.S. Provisional Application No. 60/587,085 filed on Jul. 12, 2004.

### BACKGROUND OF THE INVENTION

This invention relates to the radiation treatment of patients having treatable medical conditions.

In the last two decades, various new techniques have been developed to assess the effects of electromagnetic (EM) signals on the human body, and also to provide insight on how EM energy is absorbed by living tissue. Research has concentrated on both diagnostic and therapeutic approaches. Magneto-encephalography and, more recently, magneto-cardiography, have become useful as non-invasive tools for the diagnosis of brain and cardiac ailments. The application of a low-intensity magnetic field for the treatment of Parkinson's disease patients, or epileptic patients, has also won its due respect, as evidenced by U.S. Pat. No. 5,470,846 to Sandyk, and U.S. Pat. No. 5,496,258 to Anninos.

As far as the inventors know, nothing has been postulated regarding the possibility of treating heart failure or of abolishing or moderating cardiac arrhythmia, with the application of a low-intensity magnetic field. Nor are the inventors aware of any disclosure of the concept of using an EM radiation transducer as a regulator of atrial fibrillation or benefiting patients with hypertropic cardiomyopathy, hypertension or peripheral vascular disease.

Electric (ionic) currents excite central nervous system (CNS) neurons and cardiac myocytes, which in turn excite other member cells. The common traits of those two different groups of cells, inhibition, excitability and propagation, suggest a similar response to magnetic fields when applied to excitable tissues. The effects on heart muscle cells, or on its pacemaker cells, is expected to mimic to a certain degree the effects that the magnetic field induces in different brain neurons (cardiac pacemaker cell, and myocytes share L-type and T-type calcium channels with brain neurons).

Living beings exist continuously under the influence of magnetic fields at the Earth's surface. It constitutes what is known as the “natural magnetic field”, which exists everywhere in man's ecological niche. Other magnetic sources are called “magnetic fields of external origin”, mostly due to the activity of the Sun or from outer space. Apart from the above-mentioned natural magnetic fields, one should also take into account fields at the low frequency range of the EM spectrum, comprising frequencies below 300 Hz, and which are mostly the result of man-made technological advancements (and are mainly at 50 and 60 Hz).

In the 1950s, W. O. Schumann suggested that the space between the surface of the Earth and the ionosphere should act as a resonant cavity, somewhat like the chamber in a musical instrument. Pressing the keys on a wind instrument changes the size of the cavity and therefore changes the frequency of the standing waves within that cavity. In such musical instruments, tones are generated when the musician blows over an orifice or reed. Lightning provides energy for the Schumann resonance. While a person may be experiencing calm weather at one location on Earth, there are on average roughly 200 lightning strikes taking place each second, scattered about the planet. To use physics terminology, lightning pumps energy into the earth-ionosphere cavity, and causes it to vibrate or resonate at frequencies in the range of 7-10 Hz. This “Schumann resonance” may be modified or modulated by extra-terrestrial activities (lightning, magnetic storms on the Sun, etc.). The waves are reflected from the ionosphere, back to the Earth, back to the ionosphere, etc. This is the basis for long-distance radio communications, which are reflected by the ionosphere. The Schumann oscillations propagate for long distances and readily penetrate through the walls of buildings and into the human body. They have considerable overlap with biomagnetic fields, such as those produced by the heart and brain, except that they are thousands of times stronger.

It is also noteworthy that the membrane has a role in shielding the interior compartments of the cell from the electromagnetic field. However, the electromagnetic energy absorbed by living organisms from the outside world is generally very low; its effects on biological systems are minute, if any, and difficult to define precisely. It has been found to be harmless to human health.

Since the mode of action of WMF and VWMF radiation is done mostly through its effects on calcium channels, it is worthwhile to expand on the unique role this specific ion [Ca²⁺] has in biology by transducting, across the membrane, signals critical for cellular function.

The Importance of Calcium Ions as Biological Messengers

The living cell is a non-equilibrium, open, thermodynamic system whose boundary, the membrane, exchanges material with the outside world. This makes it possible for life to be a negentropic¹ system within a universe where entropy is constantly increasing.
¹Negentropy—a definition suggested by Erwin Schrödinger to mean negative-entropy, in his book “What is life”. It is used as entropy with a negative sign.

It now seems that information to counter entropy is not contained only in the genetic code of any particular excitable cell, but also in the different rates or frequencies at which cells transmit or respond. This is transduction through frequency encoding.

In the electrochemical version of this information processing, every action potential would be assigned a meaning based on the chemical reactions resulting from the arrival or departure of electrical charges. Such charges would be carried by ions, ligands, dipoles or electrons and thus determine the information transmitted and the movement of ions like those of calcium, which have long been known to be necessary catalysts for many intra- and extra-cellular functions. It is sort of a contrapuntal dialog between calcium ions and other ions or proteins. The cells have “cross-talk” between each other and with extra-cellular stimuli that reach them (they could be electric in nature such as discharge transfer, of electromagnetic nature, or ions), all acting to perform the common role of messengers. They communicate in a “subtle whisper” while calcium ions make for the words that enable the messages.

It is now recognized that the calcium ion (Ca²⁺) indeed has a role as one of the most important substances in cells. Ca²⁺ is a first, second and third intercellular signal transduction messenger. It is a prominent and ubiquitous messenger that can induce changes of its own and other ions on the cellular level and participates in significant functional and morphological effects.

Ion channels are abundant in the integral membrane proteins of the cells and allow the passage of specific ions through the phospholipid membrane barrier, an essential step in almost every cellular process.

Voltage-gated ion channels underlie electrical impulses in the surface membranes of excitable cells, such as neurons and muscle fibers. Na⁺, K⁺ and Ca²⁺ channels are all composed of homologous repeated domains that form a membrane-spanning pore. They are present in “signal” dependent organisms as low as bacteria, and as high as man. The channels are normally closed when transmembrane voltage is negative inside of the cell, relative to the extracellular space (resting state), but they open when the potential decreases or reverses. The fourth membrane-spanning segment (S4) within each domain contains positively charged residues and is thought to serve as the voltage sensor.

The basic functional behavior of ion channels is based on two fundamental processes: permeation and gating. Permeation is responsible for the selective and efficient translocation of ions across the membrane, whereas gating tightly controls access of ions to the permeation pathway effectively, determining selective channel activity. Ion channels, like many other proteins, have minute moving parts that perform useful functions. Distinct formations are typically characterized by differences in the relative orientations of nearby compact domains linked by hinges or swivels (linkers) composed of glycine residues or flexible loops. Segments are allowed rotation, and the implied rotations have direct bearing on the functional output since large orientation changes have been discovered in those minute cellular structures to allow them respond to resonant EM pulse.

Voltage-gated calcium channels, which are the main regulators of the flow of Ca²⁺ in excitable membranes, are composed of separate subunits (much like sodium and potassium channels). Four subunits of calcium channels have been identified: α₁, α₂−δ, β and γ.

In general, the α-subunit is known to contain the ion channel filter and has some gating properties. Within this subunit are four homologous domains containing six transmembrane helices each. The fourth transmembrane helix of each domain forms a voltage sensor. This is similar to the β-subunit of the sodium channel.

The \hat{α}-subunit is situated intra-cellularly and is involved in the membrane trafficking of α₁-subunits. The γ-subunit is a glycoprotein having four transmembrane segments. The α₂-subunit is a highly glycosylated extracellular protein that is attached to the membrane-spanning δ-subunit by means of disulfide bonds. The α₂-subunit provides structural support, whilst the δ-subunit modulates the voltage-dependent activation and steady-state inactivation of the channel.

Calcium channels are split into groups depending upon their activity or site of activity. L-type channels, for example, are found in cardiac, neuronal, endocrine and skeletal muscle tissue. T-type channels are found in the brain, in the pacemaker cells of the heart and in vascular smooth muscle.

Although electrophysiological differences do exist between the channel classes, the most obvious distinctions are between the T-type and the other types. T-type channels need only a small depolarization to be activated and are known as low-voltage activated (LVA), and they deactivate slowly. In contrast, the other classes all require a larger depolarization to be activated and are known as high-voltage-activated (HVA) channels. Although there are electrophysiological distinctions among the HVA channels, they are not sufficiently precise as to permit unambiguous differentiation solely by these criteria. Additionally, it is likely that subclasses of each of these channel types exist with different biophysical properties. At present, pharmacological differentiation is the best route for differentiating the HVA channels.

Structure and Function of the Voltage-Gated Calcium Channels

As stated, in calcium channels four homologous domains of a single polypeptide are arranged around the permeation pathway. The ion-selective permeation pathway is lined primarily by the four S6 segments and by the extracellular S5-S6 loops. The S5 and S6 segments along with the inclusive S5-S6 linker are sometimes called the pore domain of a subunit or domain. In Ca⁺ channels the main voltage sensors are the four positively charged S4 segments. Each S4 segment in the Na⁺, K⁺ channels has three to eight basic residues, either arginines or lysines, which are usually separated from each other by two neutral residues. Depolarization is expected to move S4 segments outward through the electric field. One early consequence of this S4 movement is the opening of the activation gate, believed to be formed by the cytoplasmic ends of the channel's four S6 segments, at the entrance of the permeation pathway. Prolonged depolarization also causes the inactivation of the gates, by affecting opening located elsewhere in the protein, to close (“the ball in the dock mechanism”).

Closer examination of the periodicity in the energetic perturbations within individual transmembrane segments suggests that at least major portions of all four segments (S1-S4) adopt α-helical structures. In addition, there is evidence for á-helical structure in the two extracellular linkers. The structure of α-helix in protein units of the channel is of outmost significance. It is our belief that through this principal structure, the WMF pulses in a cyclotron-resonance-mode or ion parametric mode affect the gating of the channel. An “α-helix” is a spiral configuration of a polypeptide chain in which successive turns of the helix are held together by hydrogen bonds between the amide (peptide) links.

Ca²⁺ as the charge carrier. In the presence of depolarization, extracellular Ca²⁺ will shift in an influx through the L-type channel, which brings about calcium-dependent inactivation. Inactivation is increased by raising the concentration of extra-cellular calcium [Ca²⁺]. The simplest interpretation is that the rate of inactivation can be increased or decreased at a given test potential by grading the amount of Ca²⁺ entry through individual channels.

It is yet undetermined what exact kind of secondary structural movement of helices (e.g., rotation, translation, tilting) causes the opening and closing of activation and inactivation gates of the channels, although there is some evidence that rotation plays a significant role. The biggest puzzle, however, is the way, or rather the exact mechanism, by which voltage sensor S4 movement controls gate movement and vice versa.

Yet, one must be aware that some of the so-defined “closed states” of the channel may actually not be completely closed or completely open.

Gating Involves Several Distinct Mechanisms of Activation and Inactivation

In channel function, gating is the essence of the matter providing the mechanism, which transforms information into crucial cellular action. A typical voltage-dependent channel has more than one way to open and close its pore, and these multiple gating mechanisms are important in determining the signaling behavior of the channel. In response to a positive change in the transmembrane voltage (defined as intracellular potential minus extracellular potential), the channel will open rapidly in a process called activation. Immediate return of the potential to the resting level (generally about −70 mV inside) reverses the process, closing the channel (known as deactivation). If after activation the positive potential is maintained, the channel will close despite the maintained activating stimulus; this type of closure is called inactivation. This inactivated channel is generally unresponsive to further activating stimuli, unless the membrane is returned to a negative potential, which permits the channel to recover from inactivation and return to the resting closed state.

At any rate, the opening of voltage-gated ion channels is, in most cases, followed by inactivation when the membrane is maintained at a depolarized potential. The inactivation serves a number of important functions: it terminates the action potential (Na⁺ channels), it regulates the membrane excitability (K⁺ channels), and it prevents Ca²⁺ loading in cells (Ca²⁺ channels)². Most voltage-gated ion channels have a number of different inactivation mechanisms with time constants differing with several orders of magnitudes, from microseconds to minutes.
²It is logical to assume that the effect of WMF or VWMF to induce Ca²⁺ efflux from excitable cells is executed through WMF manipulating the channel to an inactivation state.

Ca²⁺ Channels and the Heart

Voltage-dependent L-type or T-type Ca²⁺ channels play vital roles for cardiac functions, including pacemaker activity in nodal cells, trigger for Ca²⁺-induced Ca²⁺ release (CICR) effect on the sarcoplasmic reticulum (SR), and control of cardiac contractility.

The calcium current I_{CaL} also contributes to maintenance of the plateau, or elongated depolarization, of cardiac action potentials. Because I_{CaL} is important to understanding of cardiac functions in physiological as well as pathological conditions, mechanisms of its modulation have been studied extensively.

To characterize the dynamics of different ionic currents, two important issues must be considered: (a) the peak amplitude of current at depolarization, which accounts for the ionic flux through the L-type channel upon opening, and (b) the time course of current decay throughout the duration of depolarization. The latter is related to inactivation mechanisms.

One outstanding feature of I_{CaL} is that Ca²⁺ inactivates it not only by voltage but also the channel's own charge carrier. Although these two different inactivation mechanisms have been known for a long time, precise mechanisms that control I_{CaL} (Ca²⁺ current through L-type channel) during action potentials have remained uncertain, including the relative contributions of the two inactivation mechanisms. Recently, however, several studies demonstrated that Ca²⁺ entering the cell through I_{CaL} played predominant roles during the inactivation process of cardiac action potential.

The Ca²⁺ channel is not the sole current system modulated by [Ca²⁺] in the heart. Additionally, Ca²⁺ modifies other different channels, or ion-transporters, which include not only the Na⁺/Ca²⁺ exchanger (NCX) and Ikₛ potassium channel, but also the Na⁺ channel. If the localization or distribution of channel proteins is not uniform with respect to local Ca²⁺ distribution in the myocyte, modeling of the [Ca²⁺] effect on diverse channel function should be highly complicated.

It is highly logical that the effect of weak magnetic field (WMF) on calcium ionic shifts is achieved through its manipulation of L-type and T-type Ca²⁺ channels in their process of inactivation/activation.

Ca²⁺ and Arrhythmias—The Special Case of Atrial Fibrillation

The heart is a precise oscillatory organ capable of generating uninterrupted rhythmical activity over a very long period. As described before, the pacemaker cells located in the SA node generate the regular oscillatory action potentials that drive each contraction cycle. The pacemaker function depends upon the interaction between a number of plasma membrane channels, mostly T-type but also L-type calcium channels, and the Na⁺/Ca²⁺ exchanger. There is also some evidence (as mentioned before) that release of Ca²⁺ from the SR may contribute to the pacemaker potential for triggering its firing action.

Cardiac arrhythmias have been treated traditionally with anti-arrhythmic drugs that control the rhythm by altering cardiac electrical properties. However, the available drugs are not specific for atrial electrical activity and can have profound effects on ventricular electrophysiology. For example, K⁺-channel-blocking drugs that are used to treat atrial fibrillation (AF) can mimic potentially lethal congenital disorders of cardiac repolarization (prolonged Q-T syndrome that is affected by K⁺ current (Iₖ)).

Indeed it has become apparent over the past 15 years that the effects of anti-arrhythmic drugs on the electrophysiology of the ventricles can themselves paradoxically lead to life-threatening rhythm disorders (so-called “pro-arrhythmia”) and increase mortality. There has been, therefore, a shift towards non-pharmacological therapies for cardiac arrhythmias, including controlled destruction of arrhythmia-generating tissue (“ablation therapy”) and implantable devices that can sense arrhythmias and terminate them with controlled electrical discharges.

In contrast to many other cardiac arrhythmias, for which safe and highly effective non-pharmacological therapies have been developed, AF continues to be a challenge for both pharmacological and non-pharmacological approaches to treatment, which has motivated a search for improved treatment modalities. One hope is that a better understanding of the fundamental mechanisms underlying AF will lead to safer and more effective mechanism-based therapeutic approaches.

Atrial fibrillation is the single most important cause of ischaemic stroke in people more than 75 years of age. Atrial fibrillation is characterized by rapid and irregular activation of the atrium, for example, 400-500 pulses of the atrium muscular wall per minute in humans. The occurrence of AF increases with age, with a prevalence rising from 0.5% of people in their 50s to nearly 10% of the octogenarian population. Several cardiac disorders predispose to AF, including coronary artery disease, pericarditis, mitral valve disease, congenital heart disease, congestive heart failure (CHF), thyrotoxic heart disease and hypertension. Many of these are thought to promote AF by increasing atrial pressure and/or by causing atrial dilation; however, the precise mechanistic links are incompletely defined. AF also occurs in individuals without any other evidence of heart or systemic disease—a condition known as “lone AF”.

Normally, the heart rate is finely attuned to the body's metabolic needs through physiological control of the cardiac pacemaker function of the sinoatrial (SA) node (see above), which maintains a rate of about 60-90 beats per minute at rest and can fire as rapidly as 170-200 times per minute at peak exercise. During AF, atrial cells fire at rates of 400-500 times per minute.

If each atrial impulse were conducted to the ventricles, the extremely rapid ventricular rate would lead to ineffective cardiac contraction and immediate death. This is prevented by the filtering function of the atrioventricular (AV) node, which has a limited impulse-carrying capacity and through which atrial impulses must pass before activating the ventricles.

The ventricular rate during AF (the effective heart rate) is thus no longer under physiological control of the SA node, but instead is determined by interaction between the atrial rate and the filtering function of the AV node. The ventricular rate during AF is typically in the region of 100-160 pulses per minute in the absence of drug therapy. In normal individuals, a brief period of AF may cause palpitations, chest discomfort and light-headedness. Sustained AF with an uncontrolled rapid ventricular response rate can, by itself, cause severe CHF after several weeks to months, but this is reversible with proper rate and/or rhythm control if it was not stretched in time.

Owing to the loss of effective atrial contraction, and the irregular and excessively rapid ventricular rhythms that can be caused by AF, acute and sometimes life-threatening decompensation of otherwise compensated cardiac disease may occur. The loss of atrial contraction, which may curtail cardiac pump function, also leads to stasis of blood in the atrium, which promotes clot formation and the occurrence of thromboemboli, in addition to long-term dilatory effects.

The clinical approaches to AF remain limited because of inadequate efficacy and/or adverse consequences of available therapeutic avenues. The development of improved pharmacological approaches will require a better understanding of underlying ionic mechanisms. Although much has been learned over the past few years about the ionic determinants of normal human atrial repolarization, relatively little is known about how these properties are altered in patients with AF. The latter may have an important impact on the response to drugs designed to inhibit specific channels whose expression may be altered in AF.

Evolving clinical evidence shows that AF almost invariably occurs in a setting of atrial electrical dysfunction that provides a favorable basis for the arrhythmia. Inward and outward (depolarizing and repolarizing) transmembrane ionic currents are key determinants of the arrhythmia mechanisms. (I_{KI}) is the background current responsible for the considerable resting K⁺ conductance that sets the resting potential to between −70 and −80 mV. Cell firing is caused by rapid depolarization through a large Na⁺ current (I_{Na}) that brings the cell from its resting potential to a value in the region of +40 mV, providing the electrical energy for cardiac conduction. The cell then partially repolarizes through a transient outward K⁺ current (Iₜₒ), inactivation of which produces a notch in the action potential.

This is followed by a relatively flat portion of the action potential (the so-called “plateau”), which is maintained by an inward L-type Ca²⁺ current (I_{Ca}). A series of K⁺ currents that activate in a time-dependent way and show little inactivation—the so-called “delayed therapeutic devices” (I_{K})—lead to cellular repolarization.

In the human atrium, I_{K} has three components: an “ultra-rapid” component (I_{Kur}), a “rapid” component (I_{Kr}) and a “slow” component (I_{Ks}). Spontaneously automatic cells are depolarized by an inward pacemaker current (I_{f}). Na^{+/}Ca²⁺ exchange also carries an inward current during terminal repolarization and for a short time thereafter.

The balance between plateau inward and outward currents determines the action potential duration (APD): increased inward current prolongs the action potential, whereas increased outward current abbreviates it. APD governs the time from cellular depolarization to recovery of excitability at about −60 mV; the ionic current balance therefore determines the refractory period and the likelihood of re-entry.

Alterations in ionic currents that increase APD (action potential duration), and thereby the refractory period, can be used to prevent AF. For example, many clinically used drugs prolong APD and refractoriness by inhibiting I_{Kr}. They are effective in preventing AF, but can produce dangerous ventricular arrhythmias by interfering with ventricular repolarization. I_{Ks} and I_{Kur} are under strong adrenergic control, and their stimulation might contribute to AF that occurs in situations of increased adrenergic tone. Kv1.5 channels that are expressed functionally in the human atrium carry I_{Kur} but not the ventricle—inhibiting these channels may provide a means of preventing AF without the risk of ventricular pro-arrhythmia.

L-type Ca²⁺ channels deactivate rapidly when the membrane is repolarized and T-type Ca²⁺ channels deactivate relatively slowly. Ca²⁺ channel block by therapeutically useful Ca²⁺ channel antagonists is voltage dependent.

Amiodarone, bepridil, and cinnarizine block T-type Ca²⁺ channels more potently than L-type Ca²⁺ channels when binding equilibrates at normal diastolic potentials (˜−90 mV).

Biological Effects of WMF and VWMF

The interaction of electromagnetic fields with biological systems is of interest not only because of fundamental scientific curiosity, but also because of potential medical benefits.

In 1966, Reno and Beischer disclosed that when placing an isolated turtle heart in EM conditions, they found an alteration in the ion transport mechanisms at the cell membrane level, which increased the frequency of depolarization (i.e., increased the rate of cell “firing”). Schwartz et al. (1980) found that when frog hearts are exposed to a 240-Mz EM field, which was modulated at 16 Hz (the window effect), a field-dependent change was observed in efflux of Ca²⁺ ions from the cell.

It is now accepted that the effect of the magnetic field on an excitable cell's membrane works through influencing the kinetics of calcium ions (Bernardi et al., 1989). This happens in the neurons as well as in the myocytes (cardiac muscle cells).

Field intensity and modulation frequency were shown to be important determinants in WMF causing cellular Ca²⁺ efflux. Since a VWMF (extremely low-intensity magnetic field) produces significant effects, and the modulation frequency is critical for that matter, its effect which is not thermal, must be purely biological, an intervention acting at the cellular level to influence cellular functions.

First conclusion: when WMF signals cause Ca²⁺ efflux from the cells, the process is achieved through its specific coded signals and not by appreciable energy transfer. It is an information-related influence allied to the fact that the human organism maintains a variety of oscillatory/electrical activities, each characterized by a specific frequency. Indeed the existence of endogenous biological oscillatory/electrical activities makes the living organism a quasi-electromagnetic system of exquisite sensitivity.

If we succeed to decode (demodulate) its various frequency characteristics (including those of lower frequency and amplitude), we could discern some of the information carried by minute cellular mechanisms, and through interaction, alter hampered living functions.

Studies on animal neurons showed that 86% of the magnetically sensitive cells were inhibited (by the weak magnetic field) and 14% were excited. Both effects resulted from the movements of Ca²⁺ ions at the cell membrane (Azanza and del Moral, 1988). It is known that outward immigration of K⁺ ions through channels opened by Ca²⁺ fluctuations brings forth hyperpolarization of the cells wherever they exist. This is followed by efflux of the K⁺ ions (Iₜₒ) triggered by the inside shift of Ca²⁺, which may activate the cell action potential (Meech, 1978).

Thus magnetic fields induce movements of Ca²⁺ ions across the cell membrane, which affects the shifts of K⁺ ions through openings in their membrane channels. The cell may become either inhibited or excited, depending on its inherent properties and most probably also depending on the specific pattern of WMF stimulation.

One could conclude that the effect of Ca²⁺ ions under a magnetic field is, on the one hand, inhibitory, and on the other hand, excitatory. It is interesting to note that it mimics the action of caffeine on brain cells (Kuba and Nishi, 1976). Certain neurons may become excited, some inhibited. It was found that the neurons that were inhibited by caffeine were also inhibited by a magnetic field, and vice versa.

Verapamil is a typical representative of a group of Ca²⁺ channel blocking drugs, a blocker of calcium ion influx channel (L-type Ca²⁺ channel). The mechanism of the anti-anginal and anti-arrhythmic effects of verapamil is believed to be related to its specific cellular action of selectively inhibiting transmembrane influx of calcium in cardiac muscle, coronary and systemic arteries and in cells of the intracardiac conduction system. Verapamil thus blocks the transmembrane influx of calcium through the slow channel (calcium ion antagonism) without affecting, to any significant degree, the transmembrane influx of sodium through the fast channel. This results in a reduction of free calcium ions available inside cells of the above tissues.

The electrophysiological (anti-arrhythmic) effect of verapamil (by its effect on blocking the Ca²⁺ channels in the cellular membrane) is mimicking the effect induced by a pulsed magnetic field. Indeed, studying brain cells, Azanza (1989a) found that verapamil almost completely abolished the spikes (depolarization waves) of the excited neurons, thus acting as an inhibitor. Such an effect was found by the researcher to be induced by the pulsed magnetic field on 86% of the neuron population. The other 14% reacted in the opposite way and became excited (depolarized). Magnetic fields were proven to have the ability to mobilize Ca²⁺ ions from their stores in the cell membrane.

As mentioned before, among the diverse excitable cells within the heart are the highly specialized pacemaker cells (in the SA node and the AV node, which have spontaneous depolarization due to slow outward efflux of K⁺ions, until reaching the threshold of excitation). Atrial cells, and ventricular cells, all have different electrophysiological properties, yet all possess calcium channels (in addition to Na⁺ and K⁺ channels). But, in a pathological state, all may exhibit an automatic excitability of their own to fire rapidly or irregularly, causing cardiac arrhythmias. This is one mechanism of cardiac arrhythmia; the other is due to the re-entrant phenomenon where an electric (excitation) signal repeats itself by conducting in a closed circle fashion.

WMF and VWMF Stimulation and Their Possible Mechanisms of Affecting Ca²⁺ Channels

As set forth above, a weak electromagnetic field (as weak as is still capable of affecting the flux of Ca²⁺ ions across the cell membranes) affects a process that does not require an investment of cell metabolic energy. Still, it holds an ability to ignite a self-propagated process of Ca²⁺, K⁺ and Na⁺ ion shifts. It depends on the modes of WMF stimulation (frequency, intensity and configuration) and/or an additional external intervention (such as the application of drugs), to determine if the cell will discharge following its excitation or will be further inhibited.

Many of the earlier studies of calcium and WMF were looking at calcium efflux from chick brain tissue, and frequency and intensity “windows” were observed—that is, the response of the biological system depended on particular combinations of the DC magnetic flux density and the AC frequency.

By observing the response of the changing AC frequency over a wide range, it seemed likely that the active frequencies for a given DC flux density were integer multiples of a fundamental frequency. Such blinding provided the impetus for the subsequent development of the cyclotron resonance (CR) model. CR phenomena have as a basis the interaction force between a charged particle and a magnetic field. The condition for a circular movement is that the velocity or the number of turns, frequency, follows a certain relation (f=qB/2 pm). While the model has been criticized on theoretical grounds, the “harmonic” relation observed in the data seems to be real and persists over quite a spectrum of frequencies (mostly 16 Hz and its octave-harmonics).

A complex, but different, interrelation of the independent variables, AC flux density, AC frequency, and DC flux density was identified by the ion parametric resonance (IPR) model.³ Another model is the parametric resonance (PR) Model, where interference of the vibrational energy sublevels of ions, bound in calcium-binding proteins, is the basis for the interaction of weak magnetic fields with biological tissues.
³The only point of commonality between the IPR and CR models is at their fundamental frequencies. The harmonics identified by each model are inverse of each other.

None of these models have full experimental support today, but the data found in the literature show that many of the WMF biological effects seem to fulfill the basic formula for the frequency and static magnetic field. Very often, a nonlinear extremely low frequency-amplitude response is seen.

Changes in transcellular calcium concentration under a WMF effect have been reported from several laboratories using different cell models. Since calcium is a general messenger molecule, this means that the possibility exists for WMF to affect many diverse responses originating from the cell system. However it is evident that the primary interaction site for the WMF is at the membrane level, and thus, the effect is not primarily on the intra- or extracellular calcium ion per se, but on Ca²⁺ channels and most likely on their α-helical segments, which through charge transfer govern the process of activation⇄inactivation.

In vitro animal preparations exposed to WMF substantiated the assumptions that WMF has an effect on calcium efflux from cardiac (and brain) cells.

We assume that the participating channels are either the high-threshold (activated at membrane potentials nearer 0 than resting) L-type channels, or low-threshold T-type, or both. The α_{IC} subunit of the L-type channel functions around the voltage sensor, and the Ca²⁺ selective pore of the ion channel, and is responsible for its conductance.

The T (transient) type channels are activated by near-resting potential depolarization, have low-voltage activation threshold, and rapid and steady-state inactivation which occurs over a similar voltage range as activation. The T-type channel has a “window” current effect, where a limited range of voltage can open, but does not inactivate the channels. It has rapid deactivation. The T channels regulate intracellular Ca²⁺ concentration and effect rhythmic action-potential (pacemaker activity) in the heart and are blocked by amiodarone and mibefradil. The channel inactivation by mibefradil effects vasodilation of peripheral arteries and reduction of blood pressure.

### BRIEF DESCRIPTION OF THE INVENTION

The inventors have conceived of a non-invasive non-traumatic non-pharmacological cardiac therapeutic device, for regulating the human heart rhythm and affecting cardiac contraction force.

It is believed that weak magnetic fields (WMFs) and very weak magnetic fields (VWMFs)⁴ have multiple induced effects on the human cardiovascular system to rectify defective performance. If such is the case, and favorable effects are proved, its benefit over the conventional techniques would be enormous. With no need for surgical insertion into the patient, the device is handy for replacement of batteries for maintenance or regulation, can be supervised by the patient himself, who, when needed, can manipulate the controller parameters such as those affecting heart rate etc. In addition, such magnetic field cardiac therapeutic devices will have the additional advantage over invasive electrode stimulators, sometime implanted to control arrhythmias that stimulate one or two sites within the heart, whereas the magnetic field therapeutic device affects the total myocardium with the same intensity and, optionally, at the same desired point in the cardiac cycle, thus providing a WMF stimulation (optionally synchronized with the cardiac cycle) that culminates in improved cardiac function.
⁴WMF range of 0.1-200 microtesla; VWMF range of 1-100 picotesla.

It is known from in vitro experiments that WMF or VWMF can induce activation, reactivation and inhibition of the excitable cells. Biological systems in animals, as in man, can react to WMF or VWMF by having their excitable cells (brain, heart) react in resonance to a frequency-modulated magnetic field. Animal experiments suggested that WMF can affect the excitatory cell in a way similar to verapamil. It thus can have a negative cronotropic effect on cardiac pacemaker cells and can be used continuously or intermittently to alleviate atrial fibrillation. However, it is expected that manipulating the frequency or intensity of the field may also achieve a positive cronotropic effect by affecting the adrenergic nerve supply of the heart or reducing the sinus rate through stimulating cardiac vagal plexuses. The WMF or VWMF effect on excitable cells is most likely addressed through manipulating voltage-gated channels, inducing them to change their conformation moving from one certain state (activation) to another (inactivation). The rationale of such therapy is to promote organ function by preventing/reducing intra-cellular calcium overload, thus improving cardiovascular performance and patient's health.

The effect of WMF or VWMF to promote calcium efflux from atrial and myocardial cells is of utmost importance in arresting the deterioration observed with patients suffering from hypertrophic cardiomyopathy or atrial fibrillation, and not so infrequently these two disease entities are jointly expressed.

In accordance with various embodiments of the invention, the apparatus is utilized to implement a simple and safe, weak electromagnetic radiation device which radiates appropriate quantities of weak electromagnetic fields (WMF) or very weak electromagnetic fields (VWMF) during the refractory or any other period of the heart, thereby enabling essential function recovery, or ionic Ca²⁺ flux out of cardiac or smooth muscle cells and improvement of their function. A device structured according to the basic principle of the present invention modifies the magnetic coil current to achieve a desired frequency, amplitude and waveshape of the emitted radiation, changes the efficiency and specificity of radiating weak electromagnetic fields at a subject in heart failure, and optionally determines the time of WMF or VWMF firing with respect to the patient's cardiac cycle as determined by an ECG. The device utilizes a flat wide-area transducer to radiate the total organ (heart or peripheral arteries) or a target-oriented field where the focused magnetic radiation is guided by an ultrasound imaging system.

Various aspects of the invention (as recited in the independent claims) are summarized as follows.

One aspect of the present invention is a method of therapeutically treating a patient with a cardiac ailment, comprising the following steps: observing the functioning of the heart of a patient; diagnosing a cardiac condition of the patient's heart requiring therapeutic treatment; placing a plurality of electrically conductive coils near the patient's heart; and driving the coils with a voltage sufficient to cause the coils to generate a modulated magnetic field having a peak intensity, in the volume occupied by the patient's heart, less than 200 microtesla.

Another aspect of the present invention is a method of therapeutically treating a patient having a cardiac ailment, comprising the following steps: observing the functioning of a patient's heart; diagnosing a cardiac condition requiring therapeutic treatment; and applying a modulated magnetic field to the patient's heart, the modulated magnetic field having a peak intensity less than 200 microtesla in the volume occupied by the patient's heart.

A further aspect of the present invention is a system for therapeutic treatment of patients with cardiac ailments, comprising: a magnetic field transducer for transducing electrical signals into magnetic fields; a generator coupled to the magnetic field transducer for sending electrical signals thereto; an ultrasound transducer for transducing electrical signals into ultrasonic waves; and an ultrasound imaging system coupled to the ultrasound transducer and comprising a display monitor, a transmitter for sending electrical signals to the ultrasound transducer, a receiver for receiving electrical signals from the ultrasound transducer, and an image processor for converting electrical signals received from the ultrasound transducer into an image displayed on the display monitor, wherein the magnetic field transducer and the ultrasonic transducer are fixed relative to each other.

Yet another aspect of the present invention is a non-invasive pacemaker comprising: a substrate; an array of electrically conductive coils supported by the substrate; a battery power supply supported by the substrate; and a waveform generator supported by the substrate, powered by the battery power supply, and electrically coupled to the coil array.

A further aspect of the present invention is a method of reducing blood pressure in a patient, comprising the step of exposing at least portions of the patient's legs to a magnetic field having an intensity of no more than 200 microtesla.

Yet another aspect of the present invention is a device comprising: a belt of sufficient length to wrap around a chest of a patient, a multiplicity of coils supported by the belt and arranged in an area occupying only a portion of the total area of the belt; and means for fastening the belt in a position whereat the coils overlie the patient's heart.

Other aspects of the invention are disclosed and claimed below.

### BRIEF DESCRIPTION OF THE DRAWINGS

FIG. 1 is a block diagram depicting a system for the therapeutic treatment of patient's with cardiac ailments in accordance with one embodiment of the invention.

FIG. 2 is a drawing depicting a belt wrapped around a patient's chest, the belt supporting a multiplicity of coils driven by a magnetic field generator in accordance with another embodiment of the invention.

FIG. 3 is a drawing depicting a circular transducer strapped to a patient's chest and forming part of a non-invasive pacemaker in accordance with a further embodiment of the invention.

FIG. 4 is a drawing showing an array of coils incorporated in the circular transducer depicted in FIG. 3.

FIG. 5 is a drawing showing a peripheral vascular transducer in accordance with a further embodiment of the invention.

FIG. 6 is a block diagram representing circuitry incorporated in a non-invasive pacemaker in accordance with another embodiment of the invention.

FIG. 7 presents a series of graphs showing changes in ECG waveforms acquired from a pig's heart exposed to a weak magnetic field in a first experiment.

FIG. 8 presents a series of graphs showing changes in ECG waveforms acquired from a pig's heart exposed to a weak magnetic field in a second experiment.

Reference will now be made to the drawings in which similar elements in different drawings bear the same reference numerals.

### DETAILED DESCRIPTION OF THE INVENTION

In accordance with one embodiment of the present invention, magnetic fields are applied to the patient's heart through a transducer (e.g., a two-dimensional array of coils) placed over the chest. Three types of transducers can be used: (1) a flat type transducer in the form of a vest or belt to radiate the total heart; (2) a target-oriented field (TOF) transducer; and (3) a peripheral leg transducer. In the event of continuous application of pulsed EM fields, the transducer will be attached and secured to the patient's chest by a vest or belt, which may optionally contain electrodes to register the ECG signals. Upon energization of the coils with electric current, the coils produce magnetic fields that are directed into the heart, and particularly into the area of the left ventricle.

Electric current is applied to the coils by a driver comprising a voltage generator and an output resistor by which the generator is coupled to the coils. Also included in the driver is a timer for activating the generator to provide a sequence of pulses of output voltage, which are applied to the resistor. A voltmeter is connected between output terminals and the generator, to provide an indication of the magnitude of the output voltage. The coils and the resistor constitute a series circuit between the terminals of the generator. Since the internal impedance of the driver, as provided by the resistor, is several orders of magnitude greater than that of the transducer, the voltage generator acts as a current source in combination with the resistor, to provide a current to the transducer proportional to the voltage outputted by the generator. In view of the current-source function of the driver, the meter also provides an indication of the magnitude of the current flow in the coils of the transducer. The intensity of the magnetic fields produced by the current in the coils is proportional to the magnitude of the current and, accordingly, the reading of the meter serves also as an indication of the intensity of the magnetic fields applied by the transducer to the patient. The generator provides a voltage with a periodic waveform. It includes controls for selecting the AC frequency of the voltage, the waveform of the voltage, and the amplitude of the voltage. By way of example, the voltage may be a steady DC voltage, or may be varied in frequency over a range of 0.1 Hz to 10 kHz. Typically, however, in the practice of the present invention, the frequency will be in the range of 4 to 64 Hz. The waveform may be sinusoidal, triangular, trapezoidal, square, or a combination of more than one of these waveforms, i.e. a combination of sinusoidal with trapezoidal or square, and is registered by the apparatus.

In the case of energization of the coils with a sinusoidal current, the voltage generator is operated to output a peak voltage, typically, of nine volts relative to ground. This voltage provides a peak current of 20 microamperes and up to 0.5 ampere or more if needed, which is more than enough current to provide a peak magnetic field intensity of the range of from 1 picotesla to 200 microtesla. The output voltage of the generator is adjusted to provide a desired intensity to the resultant alternating magnetic fields. If desired, the resistance of the resistor may be reduced to provide still larger values of current for greater intensity of magnetic fields. Upon energization of the coils with electric current, the resultant magnetic fields have lines of force parallel to the axes of the respective coils. The locations of the coils provide that the resultant magnetic fields are uniform. In accordance with one embodiment of the invention, the intensity of the alternating magnetic fields is in the range from 1 picotesla up to 200 microtesla, and the frequency is in the vicinity of 16 Hz or its octave-harmonics.

The above-described driver will be referred to hereinafter as a “magnetic field generator”.

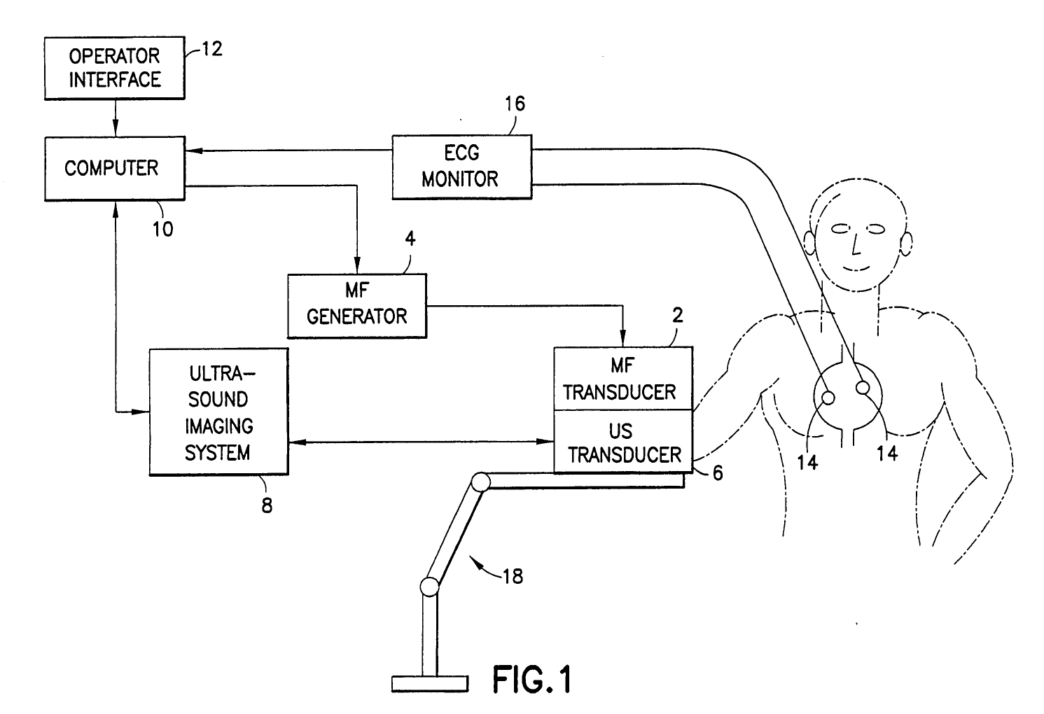

A system in accordance with one embodiment of the invention is shown in FIG. 1. The essential components of the system include a magnetic field transducer 2 for emitting electromagnetic radiation into the volume occupied by the patient's heart; and a magnetic field generator 4 for driving the magnetic field transducer 2 with electrical signals having selected waveform, intensity and frequency via electrical connectors. The intensity of the radiation should be less than 200 microtesla, and preferably should be less than 200 picotesla.

In accordance with one embodiment (not shown in FIG. 1), the magnetic field generator 4 may have manually operated input devices (such as rotary knobs or linear slides) for selecting the waveform, intensity and frequency of the electromagnetic radiation to be emitted. In accordance with the embodiment depicted in FIG. 1, the settings of the magnetic field transducer 2 are selected by a system operator via an operator interface 12 that interfaces with a computer 10, which is in turn coupled to the magnetic field generator 4, as explained in greater detail below.

Optionally, the system further comprises an ultrasound transducer 6 coupled to an ultrasound imaging system 8 via a cable. The positions and orientations of the magnetic field transducer 2 and the ultrasound transducer 6 are fixed relative to each. For example, the magnetic field transducer 2 may be mounted to the ultrasound transducer 6, or both may be mounted to the same support system. In the particular embodiment depicted in FIG. 1, the magnetic field transducer 2 is mounted to the ultrasound transducer 6, the latter in turn being mounted to the distal end of an adjustable arm 18 of a support system. The system operator can adjust the position of the arm 18 so that the focus of the ultrasound transducer 6 intersects the patient's heart, the image of which will appear on the display monitor of the ultrasound imaging system 8. Since the magnetic field transducer 2 has a fixed relationship to the ultrasound transducer 6, a graphics processor incorporated in the ultrasound imaging system 8 can overlay a graphic indicator onto the ultrasound image to indicate the center point or focal point of the magnetic field transducer.

In the embodiment depicted in FIG. 1, the magnetic field transducer 2 is of the target-oriented field (TOF) type whose effect is to produce local WMF stimulation, indicated for particular radiation regions within the heart (the SA node, the atria, the left ventricular septum, etc.). The TOF transducer 2 is mounted on and optionally interacts with a phased-array ultrasound transducer probe 6 capable of emitting and recording ultrasound pulses of 2-5 MHz. By the electronic and physical interaction of the two transducers, the apparatus according to the invention comprises means for applying WMF guided by the phased array ultrasonic sector to a focused region (about one or more square centimeters) within the heart, by means of non-planar quadruple-triangular coils or any other number and configuration of coils, to effect TOF irradiation to the required section of the selected cardiac region. In this manner, the direction of a focused magnetic field can be guided toward a desired target in the imaged heart of the patient. The TOF transducer may comprise two semicircular sections of coil with parallel current flow in the central linear section providing coil axes passing through the coils center and parallel to the central linear coil section. The plane of coil is tangential to the contour of the chest of the patient.

The TOF transducer 2 is joined with or carried on top of the ultrasound transducer probe 6. Both are held by a maneuverable arm 18 that can keep the TOF transducer 2 positioned at the patient's chest to achieve its focus directed at a selected target within the heart for a long duration of time, e.g., to allow TOF radiation for 20 minutes or more.

The ultrasound imaging system 8 may be of a conventional type that performs phased-array sector scanning in a mode that displays both tissue and blood flow. Such systems typically comprise a transmit beamformer, a receive beamformer, an image processor, a video processor and a display monitor. The ultrasound transducer 6 typically comprises one or more rows of piezoelectric transducer elements which are activated with respective time delays by the transmit beamformer to form a focused transmit beam. The returned echoes are transduced into electrical signals by the same array of piezoelectric transducer elements, which signals are respectively time delayed by the receive beamformer to form a receive beam. The ultrasound transducer 6 transmits a focused beam that is scanned over the target region, the acquired data from these transmits being processed in sequence by the image processor and the video processor to produce an image on the display monitor representing the scanned tissue and blood flow.

In accordance with a further optional feature, the operation of the magnetic field generator 4 and the ultrasound imaging system 8 can be controlled and coordinated by a computer 10 having an operator interface 12 for inputting instructions and settings. The computer may also be programmed to respond to feedback from a patient monitor, controlling the settings of the magnetic field generator as a function of data acquired by the monitor. Finally, the computer could also be programmed with software for detecting anatomical features in the ultrasound image and/or performing computations for the purpose of locating those anatomical features and then controlling the settings of the magnetic field generator accordingly. For example, the computer 10 could select the intensity or direction of the magnetic field as a function of information detected in the ultrasound image.

The majority of the ultrasound waves pass through anatomical structures and propagate onward to other structures lying further from the surface, but reflected ultrasound returns to impinge on the ultrasound transducer array of piezoelectric elements, causing those elements to compress and expand in a vibrational mode to produce electric signals which correspond to the degree of deformation. This electrical information is transformed by electronics in the ultrasound machine so that it can be displayed on a cathode-ray tube as pixel intensity data. Because the speed of sound within the body is relatively constant, the depth of the tissue interface can be known and reflected echoes are displayed on the screen on a depth scale.

The ultrasound beams transmitted into the patient's chest can be steered electronically, without moving the ultrasound transducer. Electronically steered, or “phased array” systems typically comprise 96 to 128 small piezoelectric transducer elements, which are pulsed in a very rapid, precisely controlled sequence. The top element is pulsed first; because it is very small, the ultrasound wave it generates is circular. Very soon afterwards, the second element is pulsed, and so on. The individual wavelets combine to make one compound wave that, because of the pulsing sequence, travels at an angle to the axis of the transducer array. Returning echoes do not reach all the transducer elements simultaneously; electronic circuits delay the signals from those arriving first, allowing the remainder to catch up.

Despite the necessary complexity of the electronic circuitry the phased array technique offers methods for reading the effective beam width not possible with mechanical systems. This is a very important factor in improving image quality. Focusing can be achieved by fitting a plastic lens over the face of the transducer. Phased array systems can provide additional focusing electronically; a lens works by delaying portions of the wave front and a phased array can achieve the same effect electronically by further modifying the pulsing sequence. A phased array system can also employ a technique called “dynamic focusing”. If a pulse is transmitted across two interfaces, A and B, the echo from A returns first. Its curved wave front reaches the center transducer elements before those at the edges. The electrical signals from the central elements are delayed to allow those from the edges to catch up. All the signals are then added together. A few microseconds later, echoes from B arrive. This wave front is less curved, so the delay pattern is altered. In this way the receiver changes its focal distance as echoes from more distant structures arrive, just as a pair of binoculars can be adjusted to keep an airplane in focus as it flies past. This technique rejects off-axis echoes that reduce effective beam width.

The TOF transducers, by interacting with the phased array ultrasound transducer, allow the operator to direct the focused WMF field to a selected structure of interest within the heart. Such could be right or left atria, approximate location of the S-A node or A-V node, the proximal section of the interventricular septum, and so forth.

The subcostal four-chamber view is the only satisfactory way to visualize the right atrium, and it also affords the best view of the right ventricle, since these chambers lie nearest to the transducer. Right atrial morphology can be confirmed by tilting the scan plane inferiorly so that the entrance of the inferior vena cava is seen. The standard views taken for the activation of the TOF transducer include the parastemal long/short axes and the apical, subcostal and suprasternal/subclavicular views. The patient requires no special preparation for the echographic mounted TOF treatment. The procedure is completely noninvasive and the patient is usually supine during the whole therapeutic session.

In accordance with a further option, two or more ECG electrode leads 14 are placed on the patient's body and electrically coupled to a conventional ECG monitor 16, which is in turn electrically coupled to the computer 10. Optionally, the computer 10 could control the timing of the magnetic field generation as a function of ECG waveform data acquired by the ECG monitor and transmitted to the computer 10.

In accordance with that procedure, the patient's ECG is recorded continuously—before, during, and after the application of the magnetic fields. While the ECG waveforms are being recorded and processed, electric current is applied to the coils by the magnetic field generator 2. EM signals of the desired amplitude and synchronized with the patient's cardiac cycle as reflected in the recorded ECG, will be directed at the heart during selected periods of its cycle, thereby achieving selected effects. Both the patient's ECG and the pulses of the magnetic fields are displayed on the screen of the ECG monitor 16. The magnetic field generator can manipulate the timing of the magnetic fields in temporal relation to the ECG signals from the patient.

Each one of the transducer devices can stay in action continuously or intermittently, attached to the chest wall, and the ECG signals will be acquired using a belt or an arm that will hold the transducer device in its proper place circumscribing the patient's chest.

As previously mentioned, the apparatus employed in the foregoing procedure comprises two types of cardiac transducers that impress magnetic fields upon the heart of a patient. The “area” or flat transducer comprises a two-dimensional array of coils, and is placed on the chest of the patient. The TOF transducer, guided by the ultrasound transducer, stimulates confined regions (limited in area or depth) within the heart. Upon energization of the coils with electric current, the coils produce magnetic fields that are directed into the heart, and particularly into the area of interest (the atrium, ventricles) in the patient.

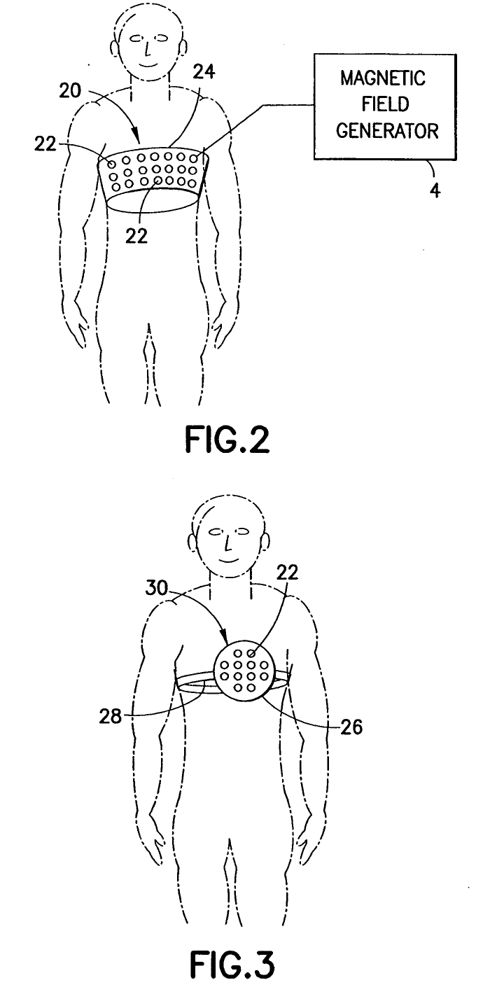

A particular construction of a magnetic field transducer 20 is depicted in FIG. 2. The transducer 20 comprises a wide belt 22 that wraps around the patient's chest and supports a multiplicity of coils in their respective. positions in a two-dimensional array. In one example, each coil has four or five turns, and has a diameter of approximately 5 mm. The belt may comprise a substrate and a cover layer, with the coil array being disposed between the substrate and the cover layer. The substrate and cover layer are formed of a flexible electrically insulating plastic material that permits flexing of the transducer to conform to the curvature of the patient's chest. The coils are formed of a flexible electrically conductive material, such as copper, which permits the foregoing flexing of the transducer.

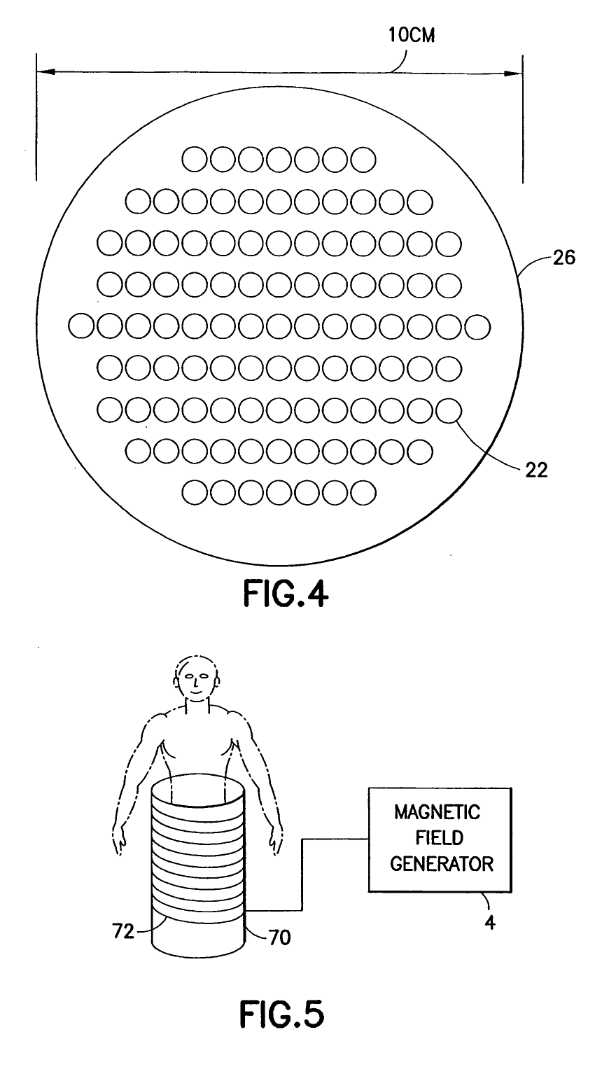

Another embodiment of a magnetic field transducer 30, suitable for use in a compact battery-powered therapeutic device, such as a pacemaker (to be described in greater detail later), is depicted in FIG. 3. The transducer 30 comprises a circular substrate 26 that may be flat or dish-shaped and that is attached to the patient's chest, e.g., by means of an elastic belt 28. The circular substrate 26 is positioned so that it overlies the patient's heart. The substrate 26 supports a multiplicity of coils 22 in their respective positions in a two-dimensional array. The belt may again comprise a substrate and a cover layer (not shown), with the coil array being disposed between the substrate and the cover layer. A specific construction of such a transducer is shown in FIG. 4. In this particular example, the circular substrate 26 has a diameter of 10 cm, while each coil has a diameter of 5 mm. The coils seen in FIG. 5 are arranged in rows and columns, but could be arranged differently.

In accordance with an alternative embodiment of the invention, a peripheral vascular transducer can be applied to the legs of the patient for the purpose of lowering the patient's blood pressure. The peripheral-vascular transducer is of similar design as the flat-area transducer except that it carries more coils to surround the patient's legs. The peripheral vascular transducer can be employed in the treatment of peripheral occlusive vascular disease, and alternatively controlling a patient's blood pressure (by inducing peripheral vasodilation). The peripheral vascular transducer (not shown in the drawings) comprises multiple arrays of coils carried in a flexible material designed in trousers-like configuration, enveloping the patient's legs. The WMF radiation of the coils, by its effect on T-type and L-type calcium channels in vascular smooth muscles, is expected to induce peripheral vasodilation.

In accordance with an alternative embodiment (depicted in FIG. 5), the peripheral vascular transducer comprises a circular cylindrical barrel 70 made of plastic or other electrically insulative material and a large coil wrapped or wound around the outside of the barrel. The coil 72 is driven by a magnetic field generator 4.

Patients who have chronic or new onset heart failure, or patients who were refractory to conventional therapy, are expected to benefit from the therapeutic treatment with weak magnetic fields. It will be imperative to fit variable properties of magnetic therapy to different cardiac arrhythmias, depending on their type and tissue (atrial, ventricular, A-V nodal, etc.). It is important to note that intracellular calcium overload facilitates cardiac dysrhythmias as well as comprising optimal cardiac function. The effect of WMF to inhibit voltage-gate calcium channels is an important move in the right direction in combating hypertrophic cardiomyopathy and heart failure.

The present invention generally relates to an application of weak electromagnetic field radiation using a method and device that can radiate weak electromagnetic fields and enable essential cardiac function recovery or activation of heart rhythm or heart contractile function in a subject. A primary object of the present invention is to use weak magnetism to generate weak electromagnetic fields so as to irradiate a target with effective radiation. In accordance with one aspect of the present invention, a weak magnetic field is transmitted to the patient's heart. This weak electromagnetic radiation is applied in a manner that causes beneficial effects on different tissues of the heart for any therapeutic purpose. In particular, the disclosed procedure can be applied for the purpose of enabling essential function recovery or activation in a subject to affect heart rhythm disturbances, or increase the contractile function of the heart by pulsing during the absolute refractory period of the ventricles, or at any other period of the cardiac cycle. It is also proposed to apply weak EM radiation for the purpose of treatment of hypertension by inducing peripheral arterial vasodilation.

The produced magnetic fields are alternating (i.e., modulated) and can be in the frequency of 0.1 Hz to 10 kHz, and their intensity can be less than approximately 200 microtesla. For clinical purposes herein, it is preferred to employ magnetic fields in the strength range of 7.5 to 100 picotesla or 0.1-200 microtesla, with an AC frequency in the range of 2 to 64 Hz. The optimal frequency depends on the specific case, yet higher intensities of the magnetic field can be selected if needed.

The affecting magnetic field pulses may optionally be synchronized with the ECG events so as to select the specific period in the cardiac cycle when different tissues may depolarize or repolarize in succession, or in some abnormal way (such as during atrial depolarization, ventricular depolarization, ventricular repolarization, or the isoelectric period when the heart relaxes its ECG activity and its mechanical performance).

If required, a pharmacological agent may be administered adjunct to WMF treatment. Following administration of the pharmacological agent, the AC pulsed magnetic fields are subsequently applied, preferably via an external magnetic coil assembly or transducer.

In addition, the main computer (item 10 in FIG. 1) further comprises a graphical user interface, including a data analysis menu, for easy operation during diagnosis of a disease. For example, the menus of “P wave isomagnetic diagram”, “irregular pulse waveform”, and “RR interval” are added, and a display parameter allows for generating a pulse wave most suitable to the diagnosis of the irregular pulse occurrence source and an equal magnetic diagram is registered (e.g., stored in memory). Further, the display conditions of single-channel waveform display for catching the irregular pulse occurrence time at a glance for the “irregular pulse waveform” menu are registered, and the display conditions of RR interval display for detection of a heartbeat error due to the autonomic nerve effect for the “RR interval” menu may be registered.

From the electrocardiogram, the P wave, QRS wave, and T wave can be ascertained. The P wave indicates the process of excitement of the atrium muscle by the stimulative wave emitted from the sinus node; the QRS wave indicates the excitement process of both the left and right ventricular muscles, and the T wave indicates the recovery process of the ventricular muscles from the previous excitement.

Thus, data analysis from the ECG provides a primary diagnosis of the heart ailment. For example, when the contour diagram of the QRS wave is considered to be wide, the display conditions are registered in the name (discrimination information) of “ventricular conduction disturbance diagnosis”. When such discrimination information is designated, the data analysis from the ECG, with its consequent patient's primary diagnosis, will automatically assist in governing the application of different options of the weak magnetic field treatment.

In accordance with an alternative embodiment of the invention, the waveform separated as the ECG signal of the heartbeat, is time-domain defined by marking the start times of the P wave, QRS wave and T wave—which times could be defined as t_{P}, t_{QRS} and t_{T}, respectively, from the top time of the signal. The times of the QRS waves are defined as t_{Q}, t_{R}, and t_{S} respectively. The timing and duration of each wave (P, QRS, T, P-R interval, QRS-T interval, R-R interval are measured and recorded.)

The predetermined time (t_{OFF}) is traced back from the point of time when the leading edge of the QRS wave matches the threshold value, and the time between the R wave of the ECG waveform and the application of the magnetic field can be determined. This is averaging, and the predetermined time is called averaging time. The ECG data may be integrated within the predetermined time range when selecting the point in time of the application of the weak magnetic field.

In accordance with a further aspect of the invention, a composition that is useful for treating heart failure, which are associated with and/or related pathogenetically to a malfunctioning heart, may be administered. The overall treatment will then be administered by application of a sufficient amount of an AC pulsed magnetic field, alone or in combination with a sufficient amount of a DC magnetic field, to the heart of a human in need of such treatment. The pharmacological composition comprises an effective amount of a composition that changes Ca²⁺ ions movement across the cell membrane of the cardiac cells of the human to be treated. A sufficient amount of an AC pulsed magnetic field of proper intensity and frequency is applied to the patient's heart, alone or in combination with a sufficient amount of a DC magnetic field of proper intensity and frequency, to treat the specific cardiac disorder. The administration of drugs prior to the application of the AC pulsed weak magnetic field is designed to sensitize the tissues and the cell membranes to the effects of the AC pulsed magnetic field.

Alone, or with prior administration of drugs, a combination of an AC pulsed magnetic field and a DC (direct current) magnetic field could also be applied simultaneously or following pretreatment with drugs to the patient's heart. The present invention thus represents a substantial advance in the treatment also of multiple cardiac conditions. The non-invasive application of a weak AC pulsed magnetic field to alleviate heart aliments have not been reported as far as the inventors are able to determine.

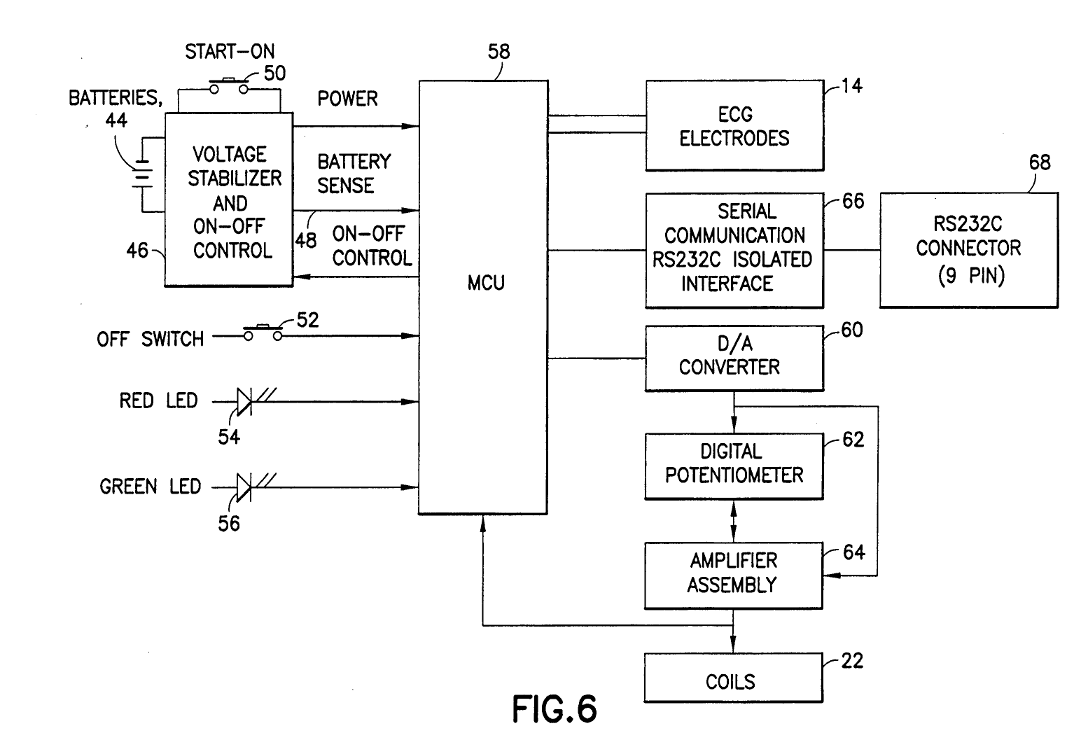

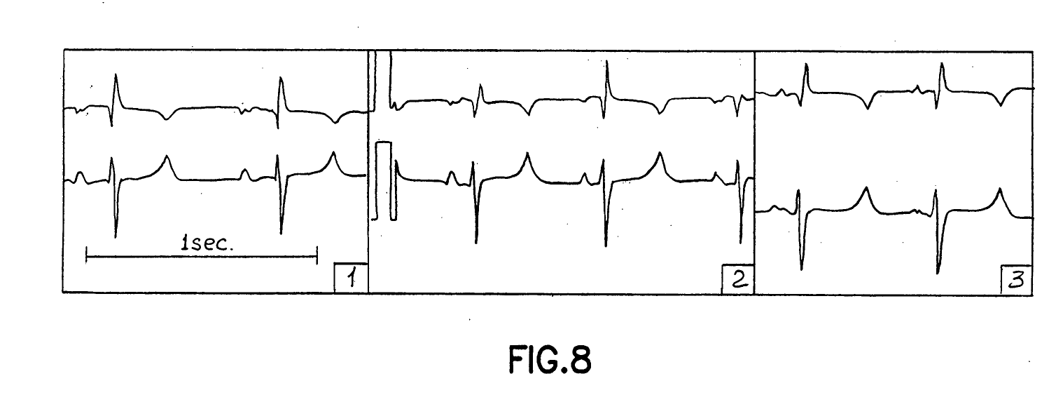

The transducer depicted in FIG. 3 can be combined with the circuitry shown in FIG. 6 to provide a non-invasive pacemaker to control sudden arrhythmias such as paroxysmal atrial fibrillation or partial atrio-ventricular (A-V) block. A first-degree A-V block is defined when the interval between the P wave and the R wave is greater than 200 msec. In experiments performed on a live pig, it was observed how irradiation with a magnetic field having an intensity of 10 pT (frequency 16 Hz) could abbreviate that interval: 2 minutes after the start of radiation, the P-R interval was reduced by about 30% (see FIG. 8, discussed later in greater detail).

The “pacemaker” device is constructed in a compact mode as a carry-on attached to a patient's chest and secured by an elastic belt. In accordance with one embodiment, the device will comprise a magnetic field generator in the form of a microprocessor or microcontroller powered by a battery, and the round configuration of a magnetic field transducer seen in FIG. 4. In addition, the ECG activity can be monitored by installing two or more ECG electrodes in the elastic belt, which leads are connected directly to the microprocessor or microcontroller. When the microprocessor receives a signal representing a cardiac disturbance from the ECG electrode(s), such as a new onset of rhythm disturbance (rapid heart rate or partial, A-V block) the microprocessor, in accordance with pre-programmed instructions, will direct the magnetic field generator to emit VWMF for a period, intensity and frequency all predetermined by the microprocessor. The device will be powered by one 3-volt battery.

FIG. 6 shows the circuitry of a battery-powered radiation treatment device in accordance with one embodiment of the invention. It is believed, but not yet demonstrated in trials, that this device is suitable for use by humans as a non-invasive pacemaker to abolish arrhythmias. The radiation treatment device comprises a microcontroller unit (MCU) 58 having an A/D input for coupling the radiation treatment device to an ECG electrode 14 attached to the chest of a patient. The microcontroller may be programmed with ECG analysis software for detecting predetermined points on the ECG waveforms acquired by the ECG electrode 14. The microcontroller 58 incorporates non-volatile memory (e.g., battery-powered memory, flash memory or other non-volatile memory technology) for storing also waveform/protocol parameters and other data received from a master or host computer. Such waveform/protocol parameters may include some or all of the following: gain, amplitude, frequency, waveshape, duration of treatment, time of treatment, number of times a treatment may be repeated, and other relevant functions, such as amplitude modulation, frequency modulation and phase modulation. These functions may be programmed to depend on the results of the ECG analysis. Alternatively, a microcomputer or microprocessor having similar functionality can be used.

The radiation treatment device comprises an RS232C communications channel by means of which waveform parameters and treatment protocol data can be loaded into the radiation treatment device from a computer, as previously described. The channel comprises serial communication RS232C isolated interface 66 and an RS232C 9-pin connector 68.

The microcontroller 58 processes the loaded treatment parameters and outputs a digital signal representing a waveform having a desired frequency and shape for driving the coils 22 of the magnetic field transducer. A digital-to-analog (D/A) converter 60 converts the digital signals output by the microcontroller 58 into an analog signal having the desired frequency and waveshape. The microcontroller 58 also outputs a digital value representing a setting to a digital potentiometer 62. The function of the digital potentiometer 62 is to adjust the level of the treatment signal, since the D/A converter 60 is always giving full amplitude. The output of the D/A converter 60 and the digital potentiometer 62 form the input signal to the amplifier assembly 64, the output of which is the current applied to the coils 22.

The microcontroller 58 outputs the digital waveform signals in accordance with the stored treatment protocol data. For example, the treatment protocol may comprise a single continuous treatment or a plurality of treatment cycles separated by quiescent intervals or rest periods.

Still referring to FIG. 6, the microcontroller 58 is powered by a battery or batteries 44. The voltage from the battery is supplied to the microcontroller 58 via a voltage stabilizer/on-off control circuit or chip 46. The voltage supplied by the battery is stabilized by the voltage stabilizer. The on-off control portion of chip 46 receives a control signal from the microcontroller 58. The treatment device can turn itself off by command from the microcontroller. The output of the analog chain (i.e., the D/A converter 60, the digital potentiometer 62 and the amplifier assembly 64) is connected into an A/D input of the microcontroller 58 to enable autotest of the proper operation of that subsystem. A Start-On pushbutton 50 is provided to turn the system on (after it is shut down). An Off pushbutton 52 is also provided for shutting down the system at any time. More precisely, the microcontroller 58 is programmed to send an Off command to chip 46 in response to pushbutton 52 being depressed. Optionally, the microcontroller can be programmed to take some other action in response to depression of pushbutton 52, in which case the latter could serve as a function switch in certain situations.

Numeral 48 indicates a low-voltage sense circuit that outputs an analog signal proportional to the current battery voltage to an input of the microcontroller 58. The microcontroller 58 incorporates an A/D converter that converts the analog signal to a digital value. That digital value is compared to a stored threshold value. When the battery voltage falls to a level corresponding to the stored threshold value, the microcontroller causes the red LED 54 to blink, indicating that the battery needs to be replaced. The red LED 54 is turned on as long as the radiation treatment device is activated. A green LED 56 is activated whenever the speaker is used and blinks when treatment is being performed. The green LED lights continuously for one minute after the end of treatment whenever number of available treatments remaining is either one or two.

The waveform parameters and treatment protocol data may be fed to the microcontroller 58 via the RS232C interface. Alternative communications channels can be employed. All parameters and protocol data are stored in a central computer and loaded into the radiation treatment device either directly or via a PC computer connected to the treatment device. The microcontroller 58 can store any desired waveform by receiving a series of values that can be repeatedly transmitted as an amplitude and time interval as selected by data transferred from the master computer. Alternatively, the microcontroller can have an internal algorithm to generate a waveform of the desired shape, amplitude and frequency to be supplied to the coils.

To test the effect of WMF on the human heart, experiments were performed on female pigs, the pig heart being closest to the human heart. In accordance with one experimental setup for radiating female pigs (50-60 kg) by WMF (range 10 picotesla to 1.8 microtesla), the animal long axis was inclined at a 5° angle relative to the Earth's magnetic field. Experiments lasted from 5 to 11 hours, while radiation time lasted up to 2 hours. Recovery time (to pre-experiment state) was 2-8 hours or more. The flat magnetic transducer placed on the pig chest was composed of 280 coils each of 1 cm diameter. The frequency, intensity and duration of activation were determined by the operator regulating the controller. ECG and blood pressure were recorded at short intervals (5-20 min) throughout the experiment. To preclude ECG changes due to vectorial shifts of the heart electrical axis, both aVL and aVF leads were recorded concomitantly. Pigs were anesthetized with Ketamine i.m and then Isofluoran. Following recovery all animals returned unharmed to their herd.

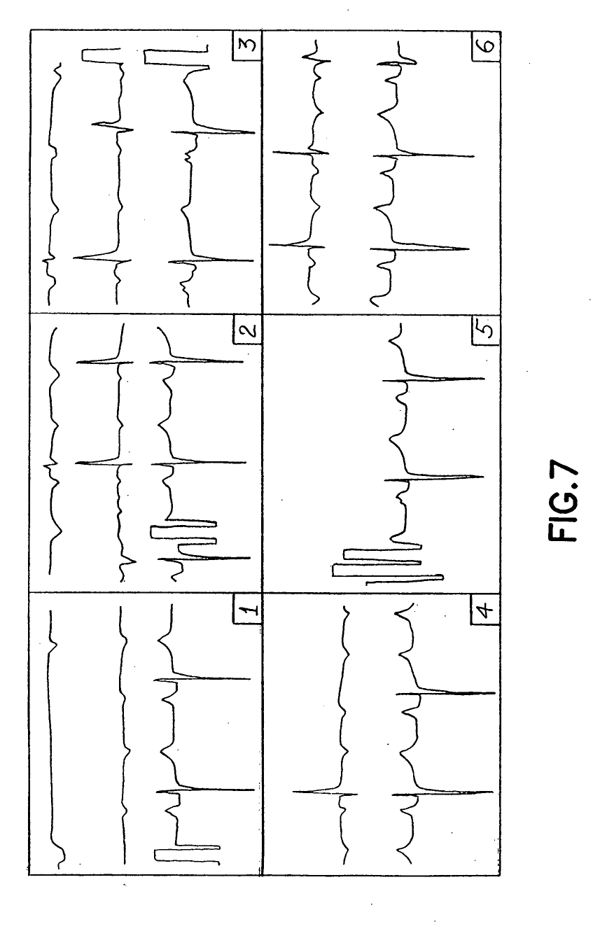

The graphs in FIG. 7 demonstrate the changes in ECG (leads aVL, aVF) post radiation for pig experiment #2, conducted on Jul. 1, 2004, (geomagnetic field 44.168 microtesla) with a field intensity of 1.8 microtesla modulated at 16 Hz. Panel (1) is control; panel (2) is a record taken after 10 min of radiation. A notched P wave could be discerned in the ECG, which represents intra-atrial conduction disturbance. Panel (3) was acquired about two hours post radiation and shows marked prolongation of the Q-T interval. Panel (4), acquired at 13:30, recovery, shows both the T wave and P wave returned to their control. Panel (5) shows that repeat radiation of 100 nanotesla induced reoccurrence of the notch in the P wave. Panel (6) was acquired at the end of experiment (15:00) and shows the ECG returned to baseline control.

The changes in the ECG throughout this pig experiment suggested inactivation of voltage-gated calcium, potassium and sodium channels. The electrocardiographic changes resemble the effect the drug Amiodarone has upon the heart.

A further experiment was conducted on Jul. 11, 2004. Records from that experiment are shown in FIG. 8. Panel (1) shows the pig's ECG before irradiation with 10-pT 16-Hz WMF; panel (2) shows immediately after the exposure to radiation; and panel (3) shows the pig's ECG 7 hours following cessation of irradiation. The pig's ECG showed abbreviation of the P-R interval and prolongation of the Q-T interval 2 min after induction of the field, 10 hours later the Q-T interval returned to the direction of normal but amplitudes of both the R and P waves were markedly diminished.

The present invention applies a weak or very weak electromagnetic field radiation at an intensity selected to enable essential recovery of cardiovascular organ function or calcium accumulation, or relaxation of their exaggerated contractile function, to be effected on the heart or peripheral vascular system. This treatment may be applied for the purposes of normalizing cardiovascular function and alleviating such ailments as cardiac arrhythmias, diastolic heart failure and hypertension.

Selected targets can be irradiated with WMF and/ or VWMF using a technique wherein the magnetic fields are activated in a selective mode wherein different sections of the target organ are radiated in a temporal and synchronized manner to achieve optimal effect. For example, the use of a flat pliable transducer that circumvents the curved surface of the organ to be radiated (chest or lower limbs) will allow, while activated, vectors of magnetic fields to be aligned with as many voltage gated Ca²⁺ and K⁺ and Na⁺ channels located at the cells membrane. Optimization of such radiation can be achieved by employing WMF radiation in multiple directions in the X, Y and Z planes and to other sections in between. Allowing the WMF or VWMF radiation to be targeted at three dimensional diverse configuration in proper space and time correlation, will enhance the probability that the magnetic field will encounter voltage-gated channels at the optimal angle relative to the channel helices to induce the cyclotron-resonance or ion parametric resonance effect or any other effect with the channel, to allow the channels to exhibit conformational changes and inactivation. The transducer will be activated serially or concomitantly according to their location within the cylindrical configuration of the pliable transducer, which is wrapped around the chest or legs or waist of the patient all to maintain optimal coverage of the irradiated target. The groups of coils will be activated intermittently in sequential mode as to avoid the interference of certain magnetic field emitted by one the group of coils with any other field emitted by another group, in respect to the X, Y and Z axes. In addition, the groups of coils in the transducer will be each activated to form WMF possessing different intensities capable of penetrating the organ at different depths in the tissue. For example, while one the group of coils may emit a WMF of 8 picotesla intensity, another group of coils may emit a field of 20 or 30 picotesla or more, all as selected by the operator, who may select to apply different ranges of field intensity. Such ranges could be 2 picotesla to 100 nT when using the very weak field or the range of 0.1-200 microtesla when using the weak field.

The groups of coils can be synchronized, one with each other, to maintain a total accurate pre-programmed performance where intensities, frequencies of the modulated signals, and temporal activation/inactivation and duration will resemble the performance of an orchestra where each performer (group of coils) knows his time of play, in relation to others, his due tone (frequency) and the intensity of his tone.

Proper stimulation by the WMF and VWMF of selected tissue or organs encompassed by the cardiovascular system is expected to induce calcium ions to shift from within the cells out, through voltage-gate ion channels as the result of the effect of the WMF or VWMF stimulation in the patient. The WMF or VWMF stimulation induces calcium ions efflux from cardiac or arterial wall cells. It is expected that WMF radiation will have positive effects on alleviating arrhythmias which are hampered by intra-cellular calcium overloading, such arrhythmias are mostly supra-ventricular and in particular atrial fibrillation. Continuous or intermittent WMF or VWMF stimulation is selected to urge recovery of deleterious cardiac function, treating cardiac supraventricular arrhythmias, hypertrophic cardiomyopathy and diastolic heart failure in the patient.

In addition, radiating peripheral arteries and arterioles with weak magnetic fields will lower excessive blood pressure in the patient by inducing relaxation in smooth muscle cells residing in the walls of arteries and arterioles of the lower limbs of the patient.

In accordance with a further aspect of the invention, a TOF transducer can be directed at a distinct target within the heart being imaged by an ultrasound imaging system, such as the right and left atria, regions of the heart's pacemaker, the proximal section of the left ventricular septum or other key areas within the heart of the patient.

In accordance with a further aspect, regular pauses in irradiation can be chosen to omit WMF pulses during short periods of time throughout the cardiac cycle, to be determined according to the ECG of the patient which after selected by the operator the ECG complexes will direct the controller and generator, to stop or start WMF radiation according to the electric phases of the patient's ECG as its signals reflect electrical events occurring across the membrane of the cardiac cells. The operator, assisted by the patient's ECG complexes, will direct the WMF radiation to be active (“on”) or not (“off”) at different phases of cardiac muscle, excitation and relaxation process, to irradiate cardiac myocytes, or cardiac pacemaker cells, at phases in their excitatory cycle such as during rapid depolarization (at the timing of the QRS complex) or the plateau (the isoelectric period between the QRS and the T wave), which is the absolute refractory period of the myocyte, or at the T wave of the ECG, which represents the relative refractory period of the cells, or radiate or pause alternatively during any other selected period during cardiac excitation, or quiescent periods as deemed by the operator. Such sequential mode of radiation of the WMF will be selected by the operator according to the phases of the cardiac action-potential if the operator wishes to synchronize myocardial stimulation with transmembrane calcium ion shifts through L-type and T-type voltage-gated calcium channels or to affect K⁺ or Na⁺ channels.

The TOF transducer can be applied to direct WMF at the section of the inter-ventricular septum of the heart where such septum is unduly hypertrophic in the patient who suffers from hypertrophic cardiomyopathy with hypertrophic obstruction to blood flow via the left ventricular outflow tract. It is expected that hypertrophic tissues in the heart that have overload of intracellular calcium ions will react to the WMF radiation by efflux of calcium from the cardiac muscle cells to alleviate hypertrophic and diastolic heart failure and release left ventricular outflow tract obstruction in hypertrophic cardiomyopathy. It is also expected that atrial cells that exhibit atrial fibrillation and that are in pathological process of electrical remodeling due to overload of intracellular calcium ions, will benefit from the effect of the WMF, by shifting excessive calcium ions from the atrial cells to the extra cellular compartment. Thus inhibiting the deleterious process of atrial remodeling which eventually leads to permanent atrial fibrillation and atrial dysfunction.

WMF can be applied to patients who suffer from permanent sinus tachycardia which eventually leads to heart failure in such the patients and by directing the TOF transducer into the region of the SA node pacemaker in the heart of such the patient, inducing the effects of the WMF directly at the region of the cardiac pacemaker (SA node) and by blocking T type calcium channels or indirectly stimulating the vagus nerve plexus within the heart, reduce the heart rate to acceptable levels.

The effect via voltage-gated calcium or sodium or potassium channels is achieved through changing such voltage-gated channels conformation to move from a state of activation to a state of inactivation or alternatively from open state to close (deactivation) state, or vice versa, to move from an inactivated state to a deactivated state proceeding to an open state.

The effect of WMF (1 microtesla, 16 Hz) was examined on in vivo pig's hearts and demonstrated changes in the ECG (see FIGS. 7 and 8), which are consistent with inactivation or blockage of the calcium and sodium and potassium channels. Such inactivation or blockage of the voltage-gated channels simulates the anti-arrhythmic effects provided by medications such as amiodarone, verapamil, flecainide and other of such classes.

Furthermore, the WMF effect on the fibrillating atria of the patients who suffer from atrial fibrillation will benefit the patients by reversing electrophysiological remodeling of the fibrillating atria by reducing calcium ions overload within the myocardial cells. WMF may be contemplated in the treatment (cardiversion) of new onset atrial fibrillation. In such case the preferred frequencies are (in addition to 16 Hz) frequencies in between 4-8 Hz, which is the range of frequencies of the atrial fibrillating waves in the patients who suffer from atrial fibrillation.

While the invention has been described with reference to particular embodiments, it will be understood by those skilled in the art that various changes may be made and equivalents may be substituted for members thereof without departing from the scope of the invention. In addition, many modifications may be made to adapt a particular situation to the teachings of the invention without departing from the essential scope thereof. Therefore it is intended that the invention not be limited to the particular embodiment disclosed as the best mode contemplated for carrying out this invention, but that the invention will include all embodiments falling within the scope of the appended claims.

As used in the claims, the term “computer” means any one of a variety of electronic devices that are capable of accepting data and instructions, executing the instructions to process the data, and outputting the results of the processing step. Examples of types of devices within the scope of this definition include, but are not limited to, a microcontroller unit, a central processing unit, a microprocessor, a microcomputer, a PC computer, a computer programmed with server software, and a laptop computer.

1. A method of therapeutically treating a patient with a cardiac ailment, comprising the following steps:
observing the functioning of the heart of a patient;
diagnosing a cardiac condition of the patient's heart requiring therapeutic treatment;
placing a plurality of electrically conductive coils near the patient's heart; and
driving said coils with a voltage sufficient to cause said coils to generate a modulated magnetic field having a peak intensity, in the volume occupied by the patient's heart, less than 200 microtesla.

2. The method as recited in claim 1, wherein the generated magnetic field has a peak intensity, in the volume occupied by the patient's heart, less than 200 picotesla.

3. The method as recited in claim 1, further comprising the following steps:
placing at least two ECG electrodes on the patient's body; and
acquiring ECG waveform data from said ECG electrodes.

4. The method as recited in claim 3, further comprising the step of synchronizing the driving of said coils with a predetermined point on an acquired ECG waveform or a predetermined point on an ECG waveform derived from one or more acquired ECG waveforms.

5. The method as recited in claim 3, further comprising the following steps:
searching acquired ECG waveform data for a predetermined set of data representing an acute or ongoing cardiac condition;
issuing an alarm signal in response to detection of said predetermined set of data representing an acute cardiac condition; and
generating the magnetic fields in response to issuance of said alarm signal.

6. The method as recited in claim 1, wherein the frequency of the modulated magnetic field is about 16 hertz.

7. The method as recited in claim 1, wherein the magnetic fields are generated in a mode wherein different sections of the patient's heart are radiated in a temporal and synchronized manner to achieve optimal effect.

8. The method as recited in claim 1, wherein the generated magnetic field is focused.

9. The method as recited in claim 8, wherein the generated magnetic field is focused in a region of the S-A node pacemaker of the patient's heart.

10. The method as recited in claim 1, wherein the magnetic fields are radiated in sequence and at multiple different focused directions.

11. The method as recited in claim 1, wherein the magnetic fields are radiated in sequence and at multiple different intensities.

12. The method as recited in claim 1, wherein different magnetic fields are automatically generated in accordance with a computer program.

13. The method as recited in claim 1, wherein the diagnosed cardiac condition is cardiac supraventricular arrhythmia or atrial fibrillation.

14. The method as recited in claim 1, wherein the diagnosed cardiac condition is hypertrophic cardiomyopathy.

15. The method as recited in claim 1, wherein the diagnosed cardiac condition is diastolic heart failure.

16. The method as recited in claim 1, wherein the diagnosed cardiac condition is sinus tachycardia.

17. A method of therapeutically treating a patient having a cardiac ailment, comprising the following steps:
observing the functioning of a patient's heart;
diagnosing a cardiac condition requiring therapeutic treatment; and
applying a modulated magnetic field to the patient's heart, said modulated magnetic field having a peak intensity less than 200 microtesla in the volume occupied by the patient's heart.

18. The method as recited in claim 17, wherein the applied magnetic field has a peak intensity less than 200 picotesla.

19. The method as recited in claim 17, wherein said magnetic field is generated by supplying a plurality of coils with electrical current, further comprising the step of placing said coils in the vicinity of the patient's heart.

20. A system for therapeutic treatment of patients with cardiac ailments, comprising:
a magnetic field transducer for transducing electrical signals into magnetic fields;
a generator coupled to said magnetic field transducer for sending electrical signals thereto;
an ultrasound transducer for transducing electrical signals into ultrasonic waves; and
an ultrasound imaging system coupled to said ultrasound transducer and comprising a display monitor, a transmitter for sending electrical signals to said ultrasound transducer, a receiver for receiving electrical signals from said ultrasound transducer, and an image processor for converting electrical signals received from said ultrasound transducer into an image displayed on said display monitor,
wherein said magnetic field transducer and said ultrasonic transducer are fixed relative to each other.

21. The system as recited in claim 20, wherein said magnetic field transducer comprises an array of electrically conductive coils.

22. The system as recited in claim 21, wherein each coil has a diameter of about 5 mm.

23. The system as recited in claim 21, further comprising a pliable substrate supporting said coils.

24. The system as recited in claim 23, further comprising elastic means for holds said substrate in a position such that said coils overlie the patient's heart.

25. The system as recited in claim 20, further comprising a computer operatively coupled to said generator and programmed to provide parameter settings to said generator.

26. The system as recited in claim 25, wherein said computer is further programmed to provide timing information to said generator.

27. The system as recited in claim 26, further comprising a plurality of ECG electrodes and an ECG monitor connected to said ECG electrodes and to said computer, said timing information transmitted to said generator by said computer being a function of ECG waveform information received from said ECG monitor.

28. The system as recited in claim 25, wherein said computer is programmed to control said generator and said magnetic field transducer to generate magnetic fields in sequence and at multiple different focused directions.

29. The system as recited in claim 25, wherein said computer is programmed to control said generator and said magnetic field transducer to generate magnetic fields in sequence and at multiple different intensities.

30. A non-invasive pacemaker comprising:
a substrate;
an array of electrically conductive coils supported by said substrate;
a battery power supply supported by said substrate; and
a waveform generator supported by said substrate, powered by said battery power supply, and electrically coupled to said coil array.

31. The pacemaker as recited in claim 30, further comprising means for attaching said substrate to a patient's chest.

32. The pacemaker as recited in claim 30, wherein said waveform generator is set to generate waveforms having an amplitude such that said coil array produces a modulated magnetic field having a peak intensity less than 200 microtesla in the volume occupied by the patient's heart.

33. The pacemaker as recited in claim 32, wherein the modulated magnetic field has a peak intensity less than 200 picotesla.

34. The pacemaker as recited in claim 30, wherein said waveform generator comprises a computer programmed to output drive signals to said coils.

35. The pacemaker as recited in claim 34, further comprising first and second ECG electrodes electrically coupled to an input of said computer, wherein said computer is programmed to output drive signals to said coils that are a function of feedback received from said ECG electrodes.

36. A method of reducing blood pressure in a patient, comprising the step of exposing at least portions of the patient's legs to a magnetic field having an intensity of no more than 200 microtesla.

37. A device comprising: a belt of sufficient length to wrap around a chest of a patient, a multiplicity of coils supported by said belt and arranged in an area occupying only a portion of the total area of said belt; and means for fastening said belt in a position whereat said coils overlie the patient's heart.

38. The device as recited in claim 37, wherein each of said coils has a diameter of about 5 mm.

39. The device as recited in claim 37, further comprising a plurality of bus lines and a multiplicity of switches for selectively connecting said coils to said bus lines
